(19) United States
(12) Patent Application Publication
(10) Pub. No.: US 2021/0251822 A1
(43) Pub. Date: Aug. 19, 2021

(54) APERTURED SUBSTRATE AND ABSORBENT ARTICLES COMPRISING THE SAME

(71) Applicant: The Procter & Gamble Company, Cincinnati, OH (US)

(72) Inventors: Koichi MORIMOTO, Beijing (CN); Gueltekin ERDEM, Beijing (CN); Meng CHEN, Beijing (CN); Li LIU, Beijing (CN); Yi YUAN, Beijing (CN); Jing ZHANG, Beijing (CN)

(21) Appl. No.: 17/178,302

(22) Filed: Feb. 18, 2021

Related U.S. Application Data
(63) Continuation of application No. PCT/CN2020/075657, filed on Feb. 18, 2020.

(51) Int. Cl. A61F 13/511; A61F 13/512

(52) U.S. Cl. CPC A61F 13/51104; A61F 2013/51178; A61F 13/5123; A61F 13/5121

(57) ABSTRACT

The present disclosure is directed to a three-dimensional substrate comprising a pattern comprising a repeat unit, wherein the repeat unit comprises a non-apertured area and a plurality of apertures defining the non-apertured area, wherein the non-apertured area comprises a protrusion, the non-apertured area having an area no less than 100 mm² as measured according to Area of Non-apertured Area; a three-dimensional substrate comprising a pattern comprising a repeat unit, wherein the repeat unit comprises at least one three-dimensional element such as protrusion and at least one land, wherein the three-dimensional element comprises at least one concave portion; and an absorbent article comprising the three-dimensional substrate disclosed herein.

### CROSS REFERENCE TO RELATED APPLICATIONS

This application is a continuation, under 35 U.S.C. § 120, of Application No. PCT/CN2020/075657, filed on Feb. 18, 2020, which is herein incorporated by reference in its entirety.

### FIELD

The present disclosure is for an apertured substrate and an absorbent article such as a baby diaper, a training pant, a feminine hygiene sanitary napkin or an adult incontinence product which comprises the apertured substrate.

### BACKGROUND

Absorbent articles for personal hygiene, such as disposable diapers for infants, training pants for toddlers, adult incontinence undergarments and/or sanitary napkins are designed to absorb and contain body exudates, in particular large quantities of urine, runny bowel movement (BM) and/or menses.

These absorbent articles comprise several layers providing different functions. A liquid permeable topsheet is disposed closest to the wearer's skin and should be capable of quickly absorbing the excreted fluid and providing breathability. Nonwoven having apertures in high density have been reported for improved fluid handling properties and breathability in the art such WO2015/134359.

A backsheet is disposed on the opposed, garment-facing side of the article. A backsheet is a portion touched and observed by the wearer or the caregiver upon use, and thus its properties are most associated with the function and quality of the article. In many absorbent articles in the market, backsheets further comprise an outermost nonwoven layer forming at least part of a garment-facing surface of an absorbent article. Much work has been done to create visual aperture patterns on nonwoven layers of absorbent articles to communicate holistic design themes to the consumer, such as attractive appearance, loftiness and pleasant tactile sense such as softness and cushiony touch in addition to functional performances such as desirable fluid handling properties, breathability, etc. depending on the desirables of the resulting article.

However, nonwoven aperturing process in general uses heat energy to set the aperture shape which negative impact nonwoven softness. Therefore, increase of apertures compromises softness of nonwoven.

Meanwhile, recent developments for disposable absorbent articles tend to focus on not only improvement of their functions e.g., superior absorbency, leakage protection and comfort, but also improvement in their aesthetic features. Aesthetic features in disposable absorbent articles are important since it may affect a consumer's perception on product premiumness as well as product functionalities, and impression and motivation to purchase. For these reasons, product aesthetics have been becoming one of important product features in the recent market of disposable absorbent articles. It is believed that superior aesthetics can provide premium impression which tends to delight the consumer and promote consumer's high motivation for purchase.

Thus, there is a need for apertured nonwovens having a clear visible apertured image or pattern without compromising softness.

In addition, there is a need for apertured nonwovens that can provide a premium impression thereby delighting consumers and promoting consumers' motivation for purchase.

### SUMMARY

The present disclosure is directed to a three-dimensional substrate comprising a pattern comprising a repeat unit, wherein the repeat unit comprises a non-apertured area and a plurality of apertures defining the non-apertured area, wherein the non-apertured area comprises a protrusion, the non-apertured area having an area no less than 100 mm² as measured according to Area of Non-apertured Area.

The present disclosure is also directed to a three-dimensional substrate comprising a pattern comprising a repeat unit, wherein the repeat unit comprises at least one three-dimensional element such as protrusion and at least one land, wherein the three-dimensional element comprises at least one concave portion.

The present disclosure is also directed to an absorbent article comprising the three-dimensional substrate disclosed herein.

The article is illustrated in the Figures as a taped diaper. For ease of discussion, the absorbent article and the acquisition-distribution system will be discussed with reference to the numerals referred to in these Figures. The Figures and detailed description should however not be considered limiting the scope of the claims, unless explicitly indicated otherwise. In particular, the invention may also be used in a wide variety of absorbent article forms, such as pant type diapers, which are pre-formed and are worn like an underwear garment, or female protection sanitary pads.

### BRIEF DESCRIPTION OF THE DRAWINGS

FIG. 1 a plan view image of an exemplary three-dimensional substrate according to the present invention.

FIG. 2 is a schematic cross-sectional view of an exemplary three-dimensional substrate according to the present invention.

FIG. 3 is a schematic cross-sectional view of an alternative three-dimensional substrate according to the present invention.

FIG. 4 is a schematic cross-sectional view of an alternative substrate according to the present invention.

FIG. 5 is an enlarged image of part of the three-dimensional substrate in FIG. 1.

FIG. 6 is a schematic illustration of a process for making a substrate as in FIG. 3.

FIG. 7 is a schematic illustration of a process for making a substrate as in FIG. 4.

FIG. 8 is a schematic plan view of an exemplary absorbent article according to the present invention.

FIG. 9 is a lateral cross-section along 2-2 of the absorbent article of FIG. 8.

FIGS. 10 and 11 are plan views of repeat units in three-dimension nonwovens.

FIG. 12˜FIG. 18 are schematic plan views of apertured patterns.

FIG. 19˜FIG. 32 are digital images of diapers having an outer cover comprising a three-dimensional substrate.

FIG. 33 is a plan view image of a three-dimensional substrate.

FIG. 34 is a plan view image of another three-dimensional substrate.

FIGS. 35A-35C are plan view images of repeat units in substrates.

### DETAILED DESCRIPTION

### Definitions of Terms

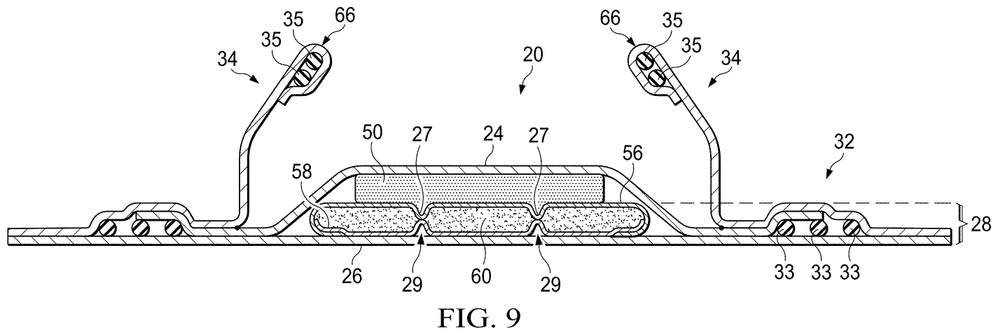

The term “absorbent article” as used herein refers to disposable products such as taped diapers, diapers having a closed waist opening (pants), feminine hygiene sanitary napkins and the like, which are placed against or in proximity to the body of the wearer to absorb and contain bodily exudates such as urine, feces and menses discharged from the body. Typical absorbent articles comprise a topsheet, a backsheet, an absorbent core, an acquisition layer and other components. A liquid permeable topsheet forms at least a portion of the wearer-facing side of the article, and a backsheet forms at least a portion, and typically the whole, of the garment-side of the article. The articles may be provided with fastening elements, such as tapes (taped diapers) or may be provided already pre-formed with a waist opening and a pair of leg openings as in an underwear (pant diapers). The absorbent articles may be for use with babies, infants, women or incontinent adults. Typical features of absorbent articles are further discussed further below, and in relation with the illustrated taped diaper in FIGS. 8 and 9, which is of course for illustration purpose only and not limiting the scope of the inventions, unless specifically indicated otherwise.

The term “macroscopic feature”, as used herein, refers to a structural feature or element that is readily visible and distinctly discernable to a human having 20/20 vision when the perpendicular distance between the viewer's eye and the web is about 12 inches (30 cm).

The term “nonwoven” as used herein refers to a manufactured material, web, sheet or batt of directionally or randomly oriented fibers, bonded by friction, and/or cohesion and/or adhesion, excluding paper and products which are woven, knitted, tufted, stitch-bonded, incorporating binding yarns or filaments, or felted by wet milling, whether or not additionally needled. The fibers may be of natural or man-made origin. The fibers may be staple or continuous filaments or be formed in situ. The porous, fibrous structure of a nonwoven may be configured to be liquid permeable or impermeable, as desired.

Any preferred or exemplary embodiments described below are not limiting the scope of the claims, unless specifically indicated to do so. The words “typically”, “normally”, “advantageously” and the likes also qualify features which are not intended to limit the scope of the claims unless specifically indicated to do so.

### Three-Dimensional Substrate

In the first invention disclosed herein, the three-dimensional substrate (herein also more simply referred to as “the substrate”) disclosed herein comprises a pattern comprising a repeat unit which comprises a non-apertured area and a plurality of apertures defining the non-apertured area, wherein the non-apertured area comprises a protrusion and a land area defining the protrusion. The non-apertured area has an area no less than 100 mm² as measured according Non-apertured Area Measurement.

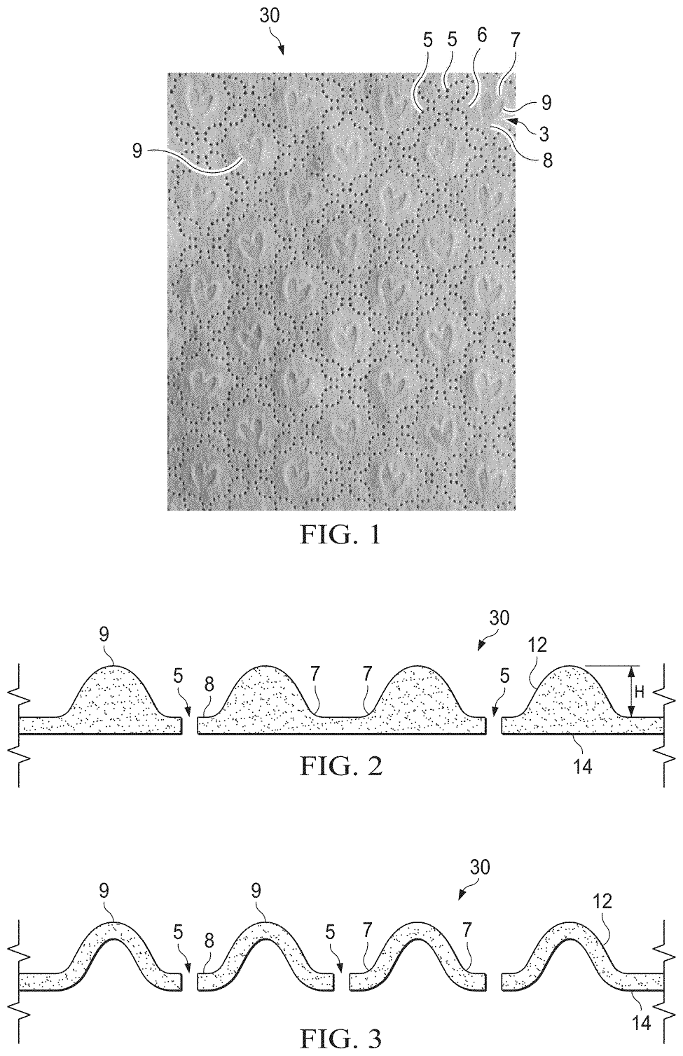

The repeat unit may repeat in a first direction. The repeat unit may repeat in a first direction and in a second direction which is different from the first direction referring to FIG. 1.

The substrate nonwoven having a pattern having a repeat unit comprising a non-apertured area and a plurality of apertures may provide a designed apertured pattern and breathability perception without compromising skin softness of nonwoven when the non-apertured area has an area no less than 100 mm² as measured according to Non-apertured Area Measurement.

In the second invention disclosed herein, the three-dimensional substrate (herein also more simply referred to as “the substrate”) disclosed herein comprising a pattern comprising a repeat unit, wherein the repeat unit comprises at least one three-dimensional element and at least one land, wherein the three-dimensional element comprises at least one concave portion.

Without being bounded by any particular theory, the substrate disclosed herein may deliver more premium perception when the repeat unit has a certain level of complexity. A proper level of complexity may be achieved when the protrusion comprises at least one concave portion, or a repeat unit has a maximum number of transitions between a land area and a protrusion no less than 6, as measured according to Protrusion—Land Transition. The substrate comprising a repeat unit comprising a protrusion comprising at least one concave portion, or a repeat unit having a maximum number of transitions between a land area and a protrusion no less than 6, as measured according to Protrusion—Land Transition may lead to perceived elegance, exquisiteness and premiumness in comparison with conventional shape of circles or ellipses or convex polygons may lead to the perceived elegance, exquisiteness and thus premium perception. A maximum number of protrusion-land transitions in a repeat unit may be increased by having one or more concaving portions in a perimeter of the protrusion. A maximum number of protrusion-land transitions in a repeat unit may be increased by forming a protrusion having a hollow internal area, i.e., internal land area substantially enclosed by a protrusion.

The substrate may be any type of web conventionally used in the art. For example, it may be a nonwoven web of natural fibers, synthetic fibers or a combination of natural and synthetic fibers. Several examples of nonwoven materials suitable for use as a substrate include, but are not limited to: spunbonded nonwovens; carded nonwovens; air through bonded carded nonwovens; spunlace nonwovens; needle punched nonwovens and nonwovens with relatively specific properties to be able to be readily deformed.

The substrate may comprise synthetic fibers, or mixed with the natural fibers. Synthetic fibers may be selected from the group consisting of polypropylene, polyethylene, polyester, polyethylene terephthalate, polybutylene terephthalate, polyamide, polylactic acid, and combinations thereof. The substrate may comprise heat fusible fibers, which may be mixed with natural fibers in the first layer of the topsheet. The term “heat fusible fibers” means fibers that when they are heated at a certain temperature, the fibers can fusion bond to other fibers that comprise the same material or different material from the heat fusible fibers.

The substrate may comprise natural fibers, such as cotton fibers, to improve the softness of the substrate, as well as to increase the amount of biodegradable material used. Using natural fibers for layers on the wearer-facing side of absorbent articles is generally desired. The substrate may thus comprise at least 15% by weight, or at least 30% by weight, or at least 50% by weight, or at least 60% by weight, or at least 75% by weight, or at least 95% by weight of natural fibers, such as cotton fibers, by weight of the substrate. The substrate may also be made of 99% to 100% by weight of natural fibers, such as cotton fibers, by weight of the substrate. Natural fibers may be selected from the group consisting of wheat straw fibers, rice straw fibers, flax fibers, bamboo fibers, cotton fibers, jute fibers, hemp fibers, sisal fibers, bagasse fibers, hesperaloe fibers, miscanthus, marine or fresh water algae/seaweeds and combinations thereof.

The substrate may be hydrophilic or hydrophobic. In order to have a hydrophobic substrate, a hydrophobic treatment may be applied to the substrate. The hydrophobic treatment may be based on synthetic material, at least to some extent, derived from natural sources. The hydrophobic treatment may be based on a natural compound, such as selected from the group consisting of natural oil, butters or waxes and combination thereof. Some examples, but not limited to, are cotton seed oil, Coconut oil, Avocado oil, Jojoba oil, Castor-seed oil, Soybean oil, Almond oil, Lanolin, Olive oil, Sunflower seed oil, Eucalyptus oil, Shea butter, Cocoa butter, Murumuru butter, Almond butter, Avocado butter, Aloe butter, Mango butter, Beeswax, Soy wax, Candelilla wax, Rice-bran wax, Coconut wax.

The substrate may typically have a basis weight from 15 g/m² to 80 g/m², or from 15 g/m² to 60 g/m², or from 20 g/m² to 50 g/m².

The three-dimensional substrate disclosed herein may be a single layer substrate or a multilayer substrate.

When the substrate comprises two or more layers, which may form a unitary structure or may remain as discrete layers which may be attached to each other by, for example, thermal bonding, adhesive bonding or a combination thereof. A unitary structure herein intends to mean that although it may be formed by several sub-layers that have distinct properties and/or compositions from one another, they are somehow intermixed at the boundary region so that, instead of a definite boundary between sub-layers, it would be possible to identify a region where the different sub-layers transition one into the other. Such a unitary structure is typically built by forming the various sub-layers one on top of the other in a continuous manner, for example using air laid or wet laid deposition.

The three-dimensional substrate may comprise discrete two layers, a first layer and a second layer. The first layer may be completely bonded to the second layer. The first layer may be intermittently bonded to the second layer. The first layer may have a basis weight of from 10 g/m² to 50 g/m², or from 15 g/m² to 40 g/m². The second layer may have a basis weight of from 5 g/m² to 50 g/m², or from 7 g/m² to 30 g/m², or from 7 g/m² to 20 g/m². The first layer is typically facing outwardly (externally) when the substrate is incorporated in an absorbent article to form, for example, a topsheet or a backsheet.

Repeat Unit

The substrate of the first invention comprises a plurality of apertures and a non-apertured area defined by the plurality of apertures. Referring to FIG. 1, a repeat unit 3 comprises a plurality of apertures 5, a non-apertured area 6, and a protrusion 9. The plurality of apertures may form a shape. In one embodiment, the protrusion comprises at least one concave portion. In another embodiment, the repeat unit has a maximum number of transitions between a land area and a protrusion at least 4, or at least 6 measured according to Protrusion—Land Transition under MEASUREMENT.

The substrate of the second invention comprises a repeat unit comprising at least one protrusion and at least one land area. Referring to FIG. 1 and FIG. 5, a repeat unit 3 comprises land area 8 and protrusion 9 comprising at least one concave portion C. In one embodiment, the repeat unit has at least 6 of a maximum transition numbers, as measured according to Protrusion—Land Transition.

### Non-Apertured Area

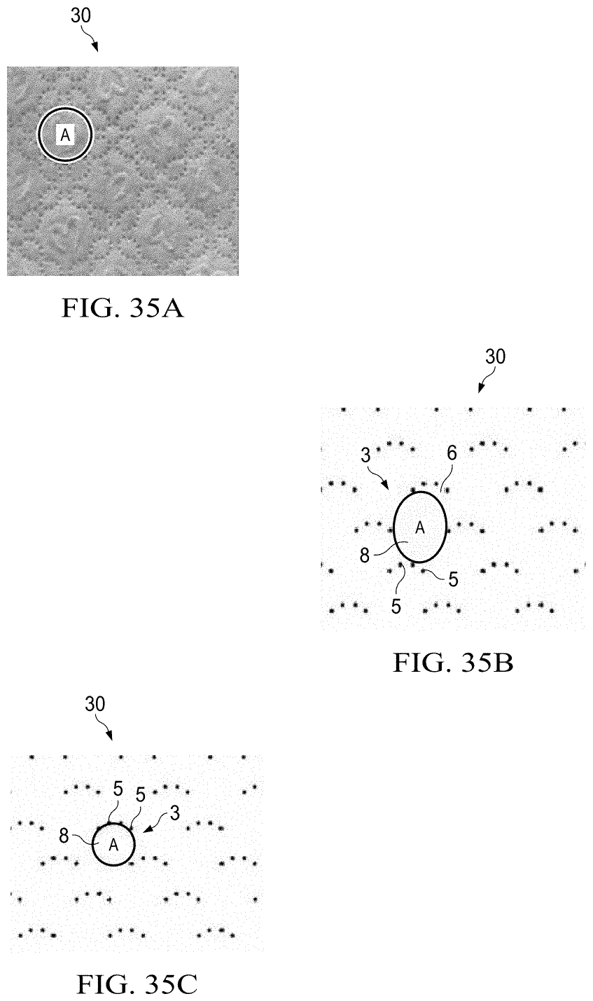

Referring to FIG. 1, a repeat unit 3 in the three-dimension substrate 30 of the first invention comprises a non-apertured area 6 defined by the plurality of apertures 5. The non-apertured area 6 comprises a land area 8 and a protrusion 9, and has an area no less than 100 mm² as measured according to Non-apertured Area under MEASUREMENT. An area A of a non-apertured area 6 intends to mean an area of a largest inscribed circle or ellipse contacting edges of at least 3 apertures, referring to FIGS. 35A-35C. FIGS. 35B and 35C omit protrusions. Though FIGS. 35B and 35C have the same aperture pattern, but may have different repeat units 3 and in turn different areas A of non-apertured area depending on locations and/or shapes of protrusions.

Without being bounded by any particular theory, the three-dimension substrate of the present invention comprising a plurality of apertures can still provide a quality softness as it has a non-apertured area defined by the plurality of apertures has an area no less than 100 mm² as measured according to Non-apertured Area Measurement, and at least one protrusion in the non-apertured area.

### Apertures

A repeat unit in the substrate of the first invention comprising a plurality of apertures which define a non-apertured area. The apertures may be regularly aligned and form a shape. The repeat unit may comprise at least 20 apertures. In the repeat unit, a distance between two adjacent apertures may be no more than about 3 mm.

Without being bounded by any particular theory, an apertured shape comprising a plurality of apertures where a distance between two adjacent apertures more than about 3 mm may make the shape highly visible.

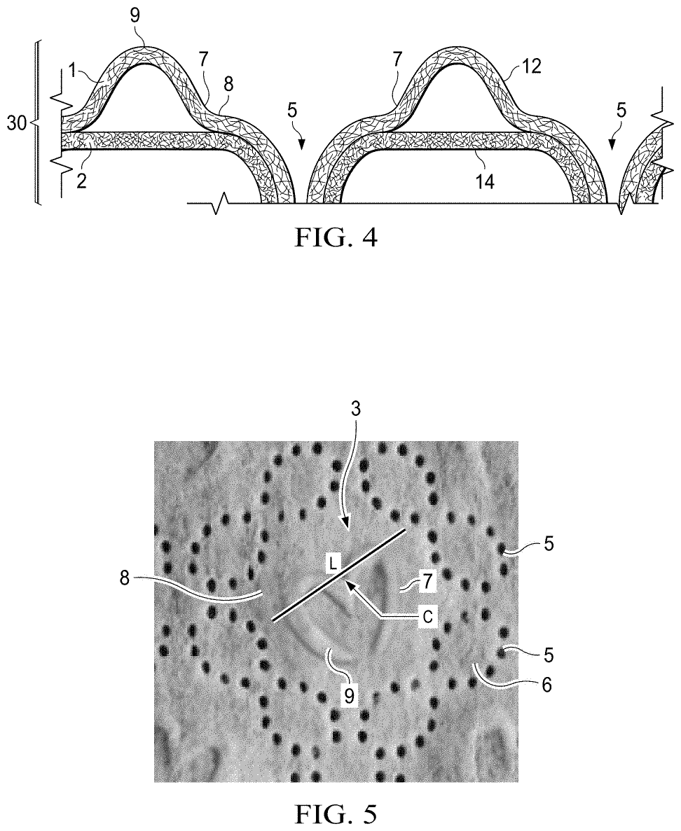

When the substrate comprises a first layer and a second layer, the first layer of the substrate may comprise a plurality of apertures. The second layer of the substrate may have a plurality of apertures at least partially, or completely aligned with the apertures of the first layer. The apertures of the first layer and of the second layer may thus be the same apertures. Referring to FIG. 4. in some embodiments, the substrate may comprise a plurality of apertures extending through the first layer and the second layer.

The apertures may be typically regularly aligned and form a shape.

The apertures may extend inwardly, away from a wear facing side or a garment-facing side and toward the absorbent core when the substrate is incorporated in an absorbent article to form, for example, a topsheet or a backsheet. The apertures may be tapered and take a conical shape such that the diameter of the aperture is larger proximate the second surface than the diameter of the opening proximate the bottom edge of the aperture. The apertures may vary in shape. For example, the shape of the apertures as seen from the first surface of the first layer may be circular, elliptic, rectangular, or polygonal. The apertures may also vary in size, such as a size of 4 mm² or less, of 3.5 mm² or less, of 3.0 mm² or less, or of 2.5 mm² or less. The size of the apertures may not be less than 0.2 mm², advantageously not less than 0.4 mm². The size is determined on the surface of the substrate which is most outwardly placed, so typically the first side of the first layer, and e.g. for conical apertures the larger aperture opening is determined.

Protrusion

A repeat unit in a pattern in the three-dimensional substrate according to the present invention comprises at least one protrusion. The protrusion is a macroscopic feature imparting a three-dimensional shape to the substrate, and may be selected from the group consisting of protrusions, recesses, and a combination thereof. The protrusion may be of any suitable configuration.

A protrusion in each repeat unit may have a same shape. The protrusion in each repeat unit may be in a same size or scale or may be a different size or scale. The protrusion in each repeat unit may be in a same orientation. In some examples, referring to FIG. 1, the protrusion in each repeat unit may a different orientation. Without being bounded by any particular theory, a three-dimension substrate disclosed herein having protrusions having an asymmetric shape may drive a better premium perception. Without being bounded by any particular theory, a three-dimension substrate disclosed herein having, protrusions arranged to have variations in orientation may enhance a premium perception.

Referring to FIG. 2, protrusions 9 may extend upwardly from land areas 8 that form a base and have an opposed distal portion from the land areas 8 forming a peak. The base of the protrusions 9, where each protrusion starts to protrude outwardly from the land areas 8, defines a perimeter 7 of the protrusion. The protrusion 9 defined by land area 8 has a perimeter 7 which provides a three-dimensional profile to the substrate 30. When the protrusion is a protrusion, referring to FIG. 2, a first side 12 of the substrate 30 has protrusions 9 and a second side 14 of the substrate 30 may be substantially flat, or may be flat. Or, referring to FIG. 3, the protrusion 9 is from the second side 14 toward the first side 12 of the substrate 30. In other embodiments, when a three-dimensional substrate comprises discrete two layers, referring to FIG. 3, the protrusion 9 may be formed on the first layer 1 only, and the area of the second layer 2 which coincides with the protrusion 9 of the first layer 1 may be substantially flat, or may be flat, so that the first layer 1 is unbonded to the second layer 2 in the protrusion 9. The first layer 1 and the second layer 2 may be joined with each other at the land areas 8, at least partially, between the protrusions 9 and/or at the apertures 5. The protrusion 9 defined by land area 8 has a perimeter 7 which provides a three-dimensional profile to the first layer 1 and more generally to the substrate 30 as a whole.

The protrusion may have a Z-directional height, H referring to FIG. 2, in the range from about 0.1 mm to about 4.0 mm, or from about 0.3 mm to about 4.0 mm, or from about 0.5 mm to about 3.0 mm, measured from a peak of the protrusion 9 to the land areas 8. Measurement of a height of a protrusion can be made on a photomicrograph according to Protrusion Height under MEASUREMENT.

In some embodiments, the protrusion comprises at least one concave portion. For example, referring to FIG. 5, a protrusion 9 has one concave portion C.

In some embodiments, two protrusions in adjacent two repeat units, one protrusion from one repeat unit and the other protrusion from an adjacent repeat unit, may be spaced apart each other at least 0.5 time, or at least 1 time longer than a length of the two adjacent protrusions. In this context, when the two adjacent protrusions have different lengths, the length of the two adjacent protrusions intends to mean a shorter length.

Two adjacent protrusions in a three-dimensional substrate of the present invention, one protrusion from one repeat unit and the other protrusion from an adjacent repeat unit, may be spaced apart each other less than 7 times, or less than 6 times, or less than 5 times shorter than a length of one of the two adjacent protrusions. In this context, when the two adjacent protrusions have different lengths, the length of the two adjacent protrusions intends to mean a longer length.

A length of a protrusion in these contexts intends to mean a longest distance between one point to another point in a protrusion.

### Land Area

A repeat unit in the three-dimensional substrate of the present invention comprises a land area. Referring to FIGS. 1-4, the land area 8 may comprise a substantially flat area.

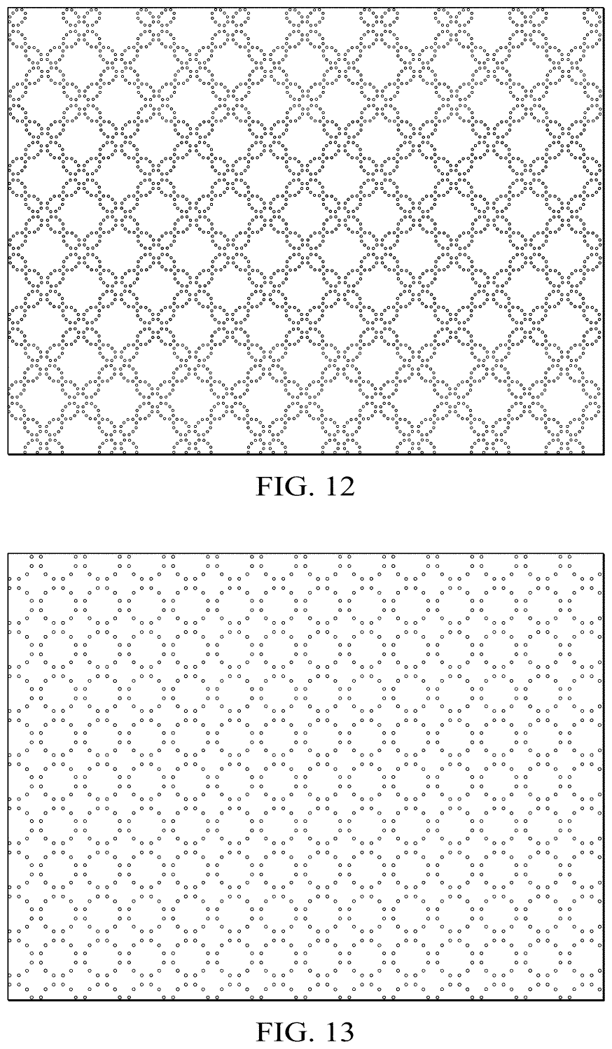

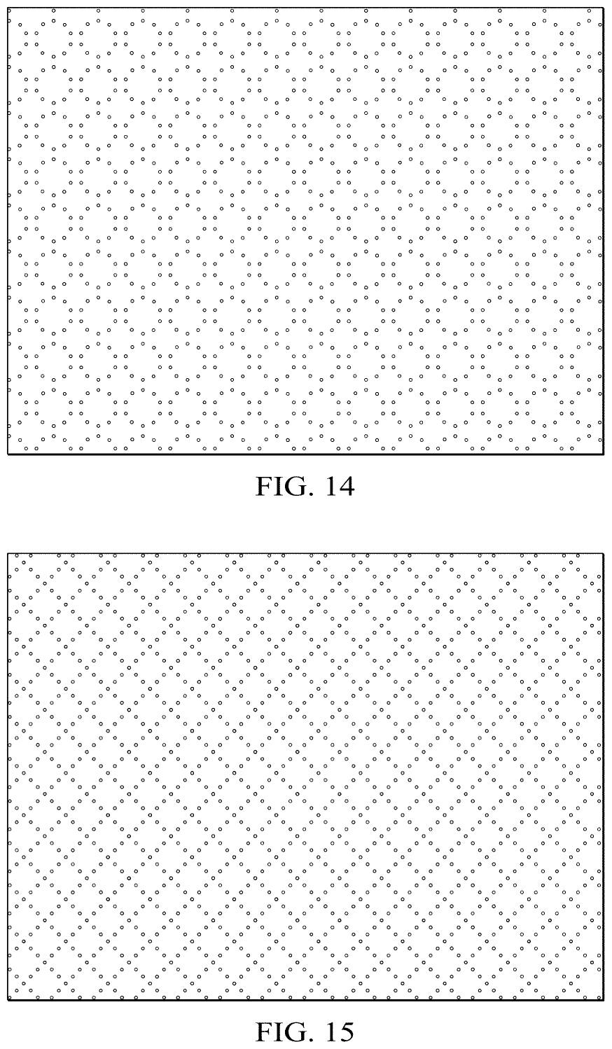

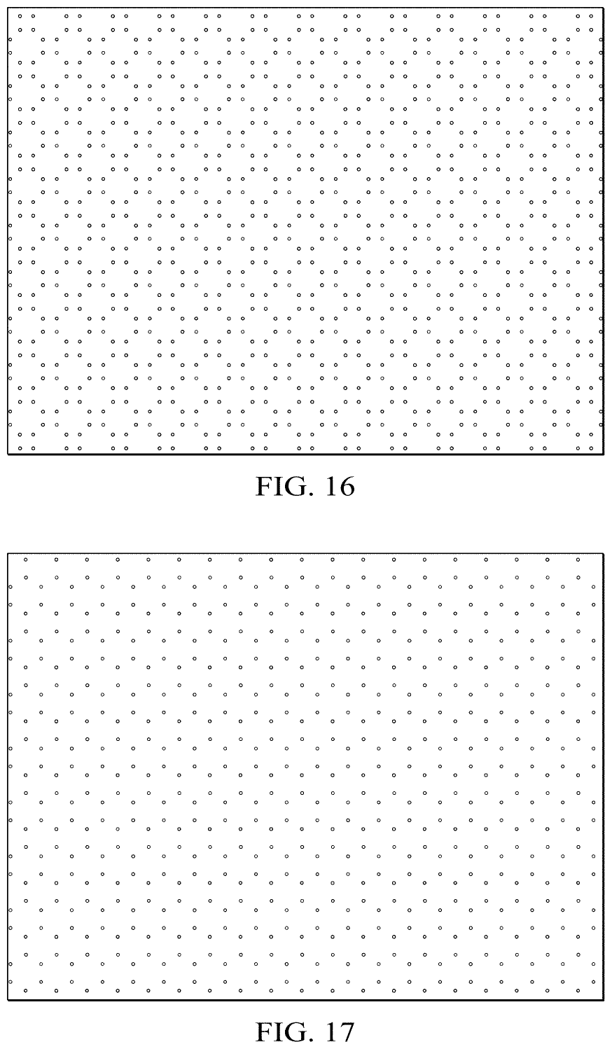

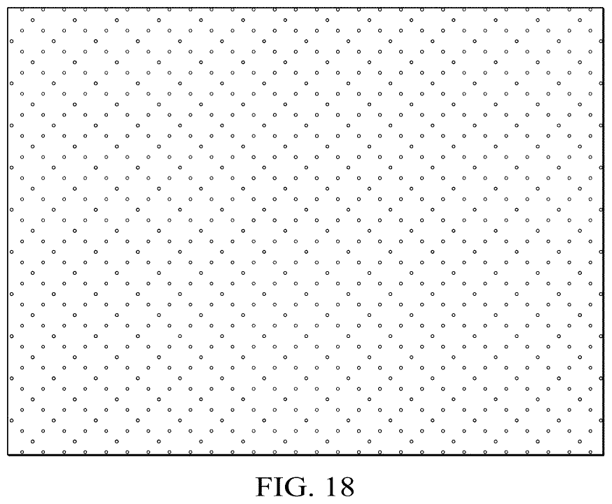

In one embodiment, the land area in a repeat unit is continuous. Referring to FIG. 1, the land area 8 in a repeat unit 3 is continuous and define the protrusion, protrusion 9 in this example. In another embodiment, the land area in a repeat unit is discontinuous. Referring to FIG. 11, land area 8 in a repeat unit 3 is discontinuous, and comprises a first region of land area 8 defining an outer perimeter of a protrusion 9 and a second region of land area 8 defining an inner perimeter of the protrusion 9. When a land area 8 is discontinuous, a land area intends to mean to include all discontinuous regions.

### Method of Making the Three-Dimensional Substrate

The three-dimensional substrate (with or without apertures) may be industrially produced by any known and suitable methods known to those of skill in the art.

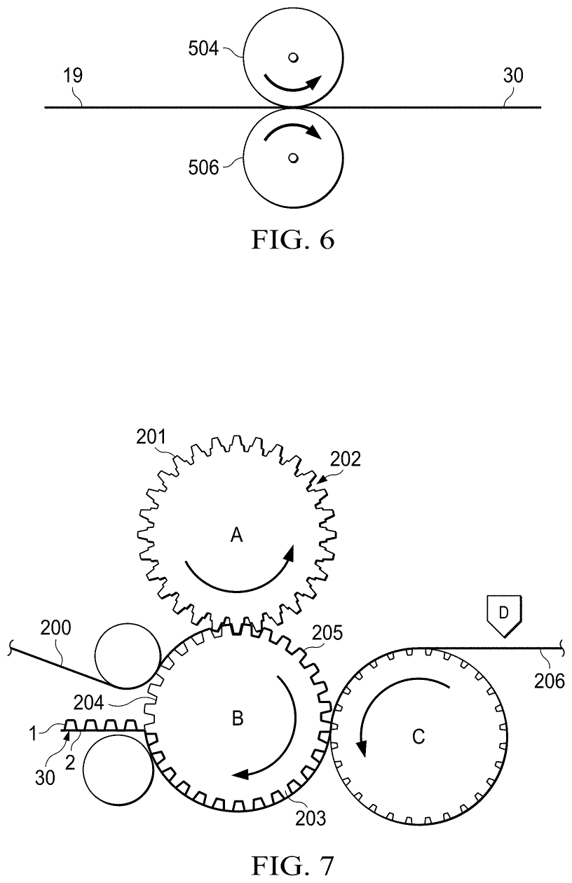

FIG. 6 is a schematic illustration of one example process for forming the substrates of the present disclosure. Referring to FIG. 6, a nonwoven 19 is passed through a nip 502 formed by a pair of rolls 500, two intermeshing rolls 504 and 506 to form a three-dimensional substrate 30. The first roll 504 comprises a plurality of first elements forming protrusion on a nonwoven 19. The first elements on the first roll 504 may be various in a size, shape, height, area, width and/or dimension which may determine the size, shape and dimension of protrusions as protrusions, embossing or a combination thereof. The first roll 504 further comprises a plurality of recesses formed in a radial outer surface of the first roll 504.

The second roll 506 comprises grooves intermeshing with the first elements in the first roll 504. The second roll 506 further comprises a plurality of second forming elements forming apertures by intermeshing with grooves the first roll 504. The rolls 504 and/or 506 may be heated.

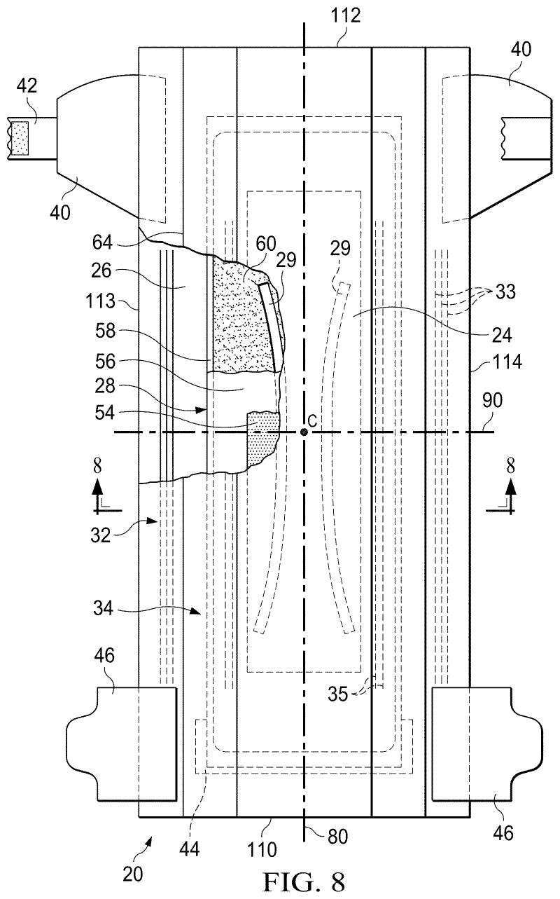

FIG. 7 is a schematic illustration of another example process for forming the substrates of the present disclosure. Referring to FIG. 7, a first layer material 200 may go through a pair of rolls named A and B. The first layer material 200 is to form the first layer 1 of the substrate.

The speed of the roll A and B may be from 5 to 600 meters/minute. The temperature range of the roll A and or the roll B may be from 40 to 200° C. The roll A may comprise a plurality of protrusions 201 extending radially outwardly from the roll A. The roll A may also comprise a plurality of recesses 202 formed in a radial outer surface of the roll A. The depth of the recesses 202 of the roll A may be from 0.5 to 10 mm, the height of the protrusions 201 of the roll A may be from 0.5 to 9 mm. The roll B may comprise a plurality of protrusions 203 extending radially outwardly from the roll B. The roll B may also comprise a plurality of recesses 204 formed in a radial outer surface of the roll B. The distal end of the plurality of protrusions 203 of the roll B may have the shape of a pin 205.

The protrusions 201 on the roll A may have a different size, shape, height, area, width and/or dimension than the protrusions 203 on the roll B. The recesses 202 formed in the roll A may have a different size, shape, height, area, width, and/or dimension than the recesses 204 formed in the roll B. The recesses 202 in the roll A may be configured to at least partially receive the protrusions 203 of the roll B, thereby creating the protrusions in the first layer material 200. The roll A may comprise a plurality of holes in the recesses area in order to receive the shape of pin 205 of the protrusions 203 of the roll B. Therefore, a plurality of apertures 5 are formed in the first layer material 200 between each two adjacent protrusions of the first layer material 200. The first layer material 200, after going through the roll A and the roll B may comprise a plurality of protrusions 9 and a plurality of apertures 5 between each two adjacent protrusions.

A second layer material 206 may be brought by a roll C. The hot-melt adhesive can be added on the first surface of the second layer material 206 by an equipment D before the second layer material 206 is in contact with the first layer material 200. The hot-melt adhesive can be advantageously uniformly sprayed on the second layer material at the basis weight indicated previously, for example 5 g/m². The roll C may comprise a plurality of holes in order to receive the shape of pin 205 of the protrusions 203 of the roll B. The second layer material 206 may pass through the roll C and the roll B and contact with the first layer material 200 at the protrusions 203 of the roll B. As the protrusions 203 of the roll B may have the shape of a pin, a plurality of apertures may be created also on the second layer material 206. The apertures in the second layer material 206 may in this way be aligned with the apertures in the first layer material 200. Pressure is applied in the land areas of the first layer and the second layer so that the adhesive forms bonds between the first and second layers to form the substrate. At the end of this process, a substrate comprising a three-dimensional, apertured first layer 1 in contact with a second layer 2 between the protrusions 9 of the first layer is obtained.

A substrate 30 for the invention but without apertures may be more easily made by first forming a three-dimensional first layer, typically by engaging a first layer material between a first and second forming members and mechanically deforming the material to form a first layer having a three-dimensional shape.

When an absorbent article comprises the three-dimensional substrate disclosed herein, the three-dimensional substrate may be disposed to form at least part of an innermost side or an outer most side of the article in such a way that the first layer is oriented outwardly relative to an absorbent article, so that the three-dimensional feature can be felt and perceived by the caretaker or a user.

### Absorbent Article

In the first invention disclosed herein, an absorbent article of the present invention comprises a liquid pervious topsheet, a liquid impervious backsheet, an absorbent core disposed between the topsheet and the backsheet, and a three-dimensional substrate disclosed herein.

Absorbent articles will now be generally discussed and further illustrated in the form of a baby diaper 1 as exemplarily represented in FIG. 8. FIG. 8 is a plan view of the exemplary diaper 1 in a flattened-out configuration with the taped ends opened and the garment-facing side turned up. An article that is presented to the user closed such as a training pant may also be represented flattened out by cutting it along its side waists. The absorbent article will typically have a front edge 110, a back edge 112 and the longitudinally-extending lateral side edges 113, 114. The front edge 110 forms the edge of the front waist and the back edge 112 of the back waist, which together when worn by the wearer form the opening for the waist of the wearer. The lateral edges 113, 114 can each form one of the leg openings. The absorbent article 20 notionally comprises a longitudinally centerline 80 dividing the article in a left side and a right side, and a perpendicular transversal centerline 90 disposed at half the length of the article as measured on the longitudinal centerline 80, with both centerlines crossing at the center point C. The taped back ends 42 attached on the front of the diaper to such as a landing zone 44.

Other layers of the absorbent article are better illustrated in FIG. 9, which shows in cross-section in addition to the liquid permeable topsheet 24 and the backsheet 26, an absorbent core 28 between the topsheet 24 and the backsheet 26.

A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers.

An optional acquisition and/or distribution layer (or system) 54 is represented in FIG. 9 together with other typical diaper components. The acquisition and/or distribution layer may comprise one layer or more than one layer. Typical acquisition and/or distribution layers may not comprise SAP as this may slow the acquisition and distribution of the fluid, but an additional layer may also comprise SAP if some fluid retention properties are wished.

The absorbent article may typically comprise a pair of partially upstanding barrier leg cuffs 34 having elastic elements 35 and elasticized gasketing cuffs 32 having elastic elements 33 substantially planar with the chassis. Both types of cuffs are typically joined to the chassis of the absorbent article typically via bonding to the topsheet and/or backsheet.

The absorbent article may comprise elasticized back ears 40 having a tape end 42 which can be attached to a landing zone 44 at the front of the article, and front ears 46 typically present in such taped diapers to improve containment and attachment.

Absorbent Core

As used herein, the term “absorbent core” refers to a component used or intended to be used in an absorbent article and which comprises an absorbent material and optionally a core wrap. As used herein, the term “absorbent core” does not include the topsheet, the backsheet and any acquisition-distribution layer or multilayer system, which is not integral part of the absorbent core. The absorbent core is typically the component of an absorbent article that has the most absorbent capacity of all the components of the absorbent article. The terms “absorbent core” and “core” are herein used interchangeably.

Referring to FIGS. 7 and 8, the absorbent core 28 can absorb and contain liquid received by the absorbent article and comprise an absorbent material 60, which may be cellulose fibers, a blend of superabsorbent polymers and cellulose fibers, pure superabsorbent polymers, and/or a high internal phase emulsion foam. The absorbent core 28 may comprise absorbent material free channels 29, through which the top side 56 of the core wrap may be bonded to the bottom side 58 of the core wrap. The core wrap bonds 27 may at least persist as the absorbent core 28 swells upon liquid absorption and creates three-dimensional channels at the wearer-facing surface of the article. Of course, this is entirely optional, the absorbent core may also not have bonded channels, or even unbonded channels. The absorbent material defines an absorbent material area 8, which may be rectangular as show in in FIG. 8, but it is also common to have a shaped area which is tapered in the area around the transversal centerline 90.

The absorbent material comprises a liquid-absorbent material commonly used in disposable absorbent articles such as comminuted wood pulp, which is generally referred to as airfelt or fluff. Examples of other suitable liquid-absorbent materials include creped cellulose wadding; melt blown polymers, including co-form; chemically stiffened, modified or cross-linked cellulosic fibers; tissue, including tissue wraps and tissue laminates, absorbent foams, absorbent sponges, superabsorbent polymers (herein abbreviated as “SAP”), absorbent gelling materials, or any other known absorbent material or combinations of materials.

The absorbent material in the absorbent core can be any type. It can be an airfelt core comprising wood cellulose fibers such as pulp fibers mixed with SAP, or an airfelt-free core free from such cellulose fibers. Airfelt cores typically comprises from 40% to 80% of SAP. For absorbent cores comprising a relatively high proportion of SAP at least partially enclosed within the core wrap, the SAP content may represent in particular at least 80%, 85%, 90%, 95% and up to 100%, of superabsorbent polymer by weight of the absorbent material. The absorbent material may in particular comprise no or only small amount of cellulose fibers, such as less than 20%, in particular less than 10%, 5% or even 0% of cellulose fibers by weight of the absorbent material. The absorbent core may comprise an absorbent material comprising at least 80%, at least 90%, at least 95%, or at least 99% by weight of the absorbent core. The term “superabsorbent polymer” refers herein to absorbent material, which may be cross-linked polymer, and that can typically absorb at least 10 times their weight of an aqueous 0.9% saline solution as measured using the Centrifuge Retention Capacity (CRC) test (EDANA method WSP 241.2-05E). The SAP may in particular have a CRC value of more than 20 g/g, or more than 24 g/g, or of from 20 to 50 g/g, or from 20 to 40 g/g, or from 24 to 30 g/g. The SAP may be typically in particulate forms (superabsorbent polymer particles), but it not excluded that other forms of SAP may be used such as a superabsorbent polymer foam for example.

Backsheet

An absorbent article according to the present invention comprises a liquid impervious backsheet. The backsheet may be designed to prevent the exudates absorbed by and contained within the absorbent article from soiling articles that may contact the absorbent article, such as bed sheets and undergarments. The backsheet may be substantially water-impermeable. Suitable backsheet materials may include breathable materials that permit vapors to escape from the absorbent article while still preventing exudates from passing through the backsheet. The backsheet may comprise a liquid impermeable film. The backsheet may comprise a wetness indicator.

Outer Cover

An absorbent article according to the present invention may comprise an outer cover forming at least part of a garment-facing surface of the absorbent article. The outer cover may comprise a three-dimensional substrate disclosed herein in such a way that a first side having protrusions of the substrate forms at least part of the garment-facing side of the article. When a backsheet comprises a liquid impermeable polymer film, the polymer film and the substrate disclosed herein may be disposed in a face to face relationship in such a way that the substrate is towards the garment-facing side of the article, and the film is towards an absorbent core of the article. The first layer is oriented outwardly relative to the article, so that the protrusions can be felt by the caretaker or a user feeling the garment-facing side of the article.

The absorbent article may also comprise other typical components, which are not represented, such as a back-elastic waist feature, a front elastic waist feature, transverse barrier cuff(s), a lotion application, etc.

Components of the disposable absorbent article described in this specification can at least partially be comprised of bio-sourced content as described in US 2007/0219521A1 Hird et al published on Sep. 20, 2007, US 2011/0139658A1 Hird et al published on Jun. 16, 2011, US 2011/0139657A1 Hird et al published on Jun. 16, 2011, US 2011/0152812A1 Hird et al published on Jun. 23, 2011, US 2011/0139662A1 Hird et al published on Jun. 16, 2011, and US 2011/0139659A1 Hird et al published on Jun. 16, 2011. These components include, but are not limited to, topsheet nonwovens, backsheet films, backsheet nonwovens, side panel nonwovens, barrier leg cuff nonwovens, super absorbent, nonwoven acquisition layers, core wrap nonwovens, adhesives, fastener hooks, and fastener landing zone nonwovens and film bases. In at least one embodiment, a disposable absorbent article component comprises a bio-based content value from about 10% to about 100% using ASTM D6866-10, method B, in another embodiment, from about 25% to about 75%, and in yet another embodiment, from about 50% to about 60% using ASTM D6866-10, method B. In order to apply the methodology of ASTM D6866-10 to determine the bio-based content of any disposable absorbent article component, a representative sample of the disposable absorbent article component must be obtained for testing. In at least one embodiment, the disposable absorbent article component can be ground into particulates less than about 20 mesh using known grinding methods (e.g., Wiley® mill), and a representative sample of suitable mass taken from the randomly mixed particles.

### Measurement

### 1. Aperture Configuration

An aperture diameter and a distance between two adjacent apertures are measured by scale loupe 15× sourced from PEAK.

When an aperture is an oval, an aperture diameter intends to mean a smallest diameter. When an aperture is a polygon, an aperture diameter intends to mean a longest distance between two points on the polygon smallest diameter.

A distance between two adjacent apertures intends to mean a shortest distance between an edge of an aperture to an edge of an adjacent aperture.

### 2. Area of Non-Apertured Area

An area of a non-apertured area is measured using image analysis.

A repeat unit image is generated under uniform surface lighting condition, using an optical microscope such as Keyence 3D Measurement System VR-3200 or equivalent. The magnification and focus is adjusted such that at least one repeated unit with complete non-aperture area of the substrate specimen is suitably captured for measurement. The microscope acquires a specimen image with at least one repeated unit with complete non-aperture area.

Analyses are performed using ImageJ software (version 1.49v or above, National Institutes of Health, USA) and calibrated against a ruler certified by NIST or equivalent. The image needs to be distance calibrated with an image of the ruler to give an image resolution, e.g. 67.8 pixels per mm Open a specimen image in ImageJ. Set the scale according to the image resolution. Use elliptical selection tool to select the maximum circle or ellipse area connecting inner edges of apertures that connects edges of at least 3 apertures. After selected a maximum circle or ellipse, measure the area inside of the selected ellipse by using measurement tool at nearest 0 mm².

### 3. Diaper Photorealistic Image Generation

Photorealistic images of diapers reflecting different design options are generated using the methodologies of digitally synthesizing and manipulating visual content well known in the area of computer graphics and 3D rendering such as Norman I Badler, Andrew S. Glassner, 3D Object Modeling Lecture Series, University of North Carolina at Chapel Hill. A high-resolution 3D model of diaper is first developed using polygonal modeling process or other ways to provide the geometry information for rendering. In the rendering process, additional information including texture, viewpoint, lighting and shading is then acquired to generate the final appearance, providing simulated product images.

A rendering plug-in of V-Ray for Autodesk 3ds Max or equivalent 3D computer graphics software is used to apply a ray-tracing algorithm for producing images with more photorealism, by tracing the path of light through each pixel in an image plane and simulating the effects of its encounters with virtual objects. Multiple layers of materials with preferred rendering parameters, representing the product components like backsheet film and outer-cover nonwoven, are superposed and virtually mixed to generate the prototyping results. Changes in visual attributes comprising surface texture design (e.g. patterns of protrusions or apertures surrounded by land areas), graphics and color printing, as well as material optical properties such as reflection and opacity, are implemented through rendering by editing the material features of texture-mapping, bump-mapping, reflection, opacity, shading, shadowing or other variables.

### 4. Protrusion—Land Transition

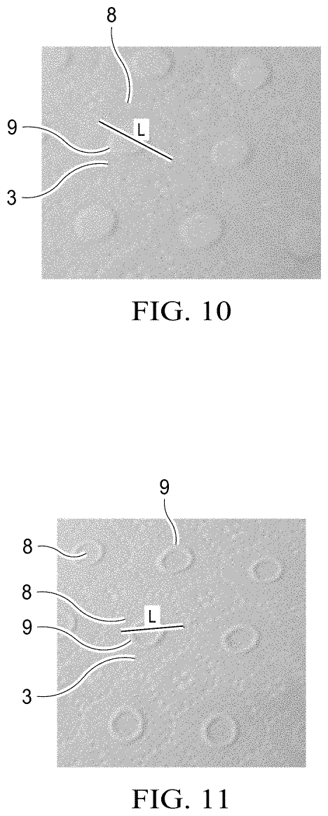

Referring to FIG. 5, draw a straight line L in a repeat unit 3 across one protrusion, protrusion 9 in this example, to encounter the protrusion as many times as possible and count numbers of transitions between the protrusion 9 and a land area 8. In FIG. 5, a repeat unit 3 has a protrusion 9 and a land area 8 defining protrusion 9, and has a maximum transition numbers of 6. Referring to FIGS. 10 and 11, repeat unit 3 in FIG. 10 has a maximum numbers of transitions of 2 and repeat unit 3 in FIG. 11 has a maximum number of transition of, respectively.

### 5. Protrusion Height

Sample Preparation

When a three-dimensional substrate is available in its raw material form, a 80 mm×30 mm specimen with one of the long edges cutting across at least one protrusion, is excised from the raw material using a razor blade. When a specimen is obtained from an absorbent article, a test specimen of the substrate is first carefully removed from the absorbent article in a manner that avoids imparting any contamination or distortion during the process. A cryogenic spray (such as Sunto™ Freeze Spray, Sunto (HK) International, China) may be used to remove the specimen. If necessary, the substrate specimen may be excised together with the underling material layer to keep its original three-dimensional structure. For cross-section imaging, the specimen is mounted vertically on a sample-holding plate via double-sided tape in a manner that the long edge across the protrusion is horizontally positioned, while the first side of the substrate is away from the plate and the specimen vertically exceeds the plate edge by about 3 mm

Height Measurement

The height measurements of protrusions are performed on the cross-section images generated under uniform surface lighting condition, using an optical microscope such as Keyence 3D Measurement System VR-3200 or equivalent. The magnification and focus is adjusted such that the cross-section side of the substrate specimen is suitably enlarged for measurement.

Analyses are performed using ImageJ software (version 1.49v or above, National Institutes of Health, USA) and calibrated against a ruler certified by NIST or equivalent. The image needs to be distance calibrated with an image of the ruler to give an image resolution, e.g. 67.8 pixels per mm. The microscope acquires a specimen image with at least one protrusion along the cross-section side.

Open a specimen image in ImageJ Set the scale according to the image resolution. The height of a protrusion is measured as the vertical distance between the peak i.e. the highest point and the surface of surrounding land area for one element. The measurement is repeated on five different protrusions to calculate the arithmetic mean to the nearest 0.1 mm.

### EXAMPLES

### Example 1. Clarity Test of Apertured Shape

7 Nonwoven images with patterns of FIGS. 12-18 respectively and dimensions indicated in Table 1 below were prepared. 18 panelists of age 20 to 40 in mix 11 females and 7 males were recruited as panels. The panels were exposed to the nonwoven images printed on the paper in 1:1 scale in randomized order one by one, and requested rating shape clarity of each image exposure on scales 0 (poor), 25 (fair), 50 (good), 75 (very good) and 100 (excellent). The results of average rating were indicated in the Table 1 below.

TABLE 1

| | | Area of Non- | Aperture | Distance* between | | |
|---|---|---|---|---|---|---|
| | | apertured | diameter | apertures | | |
| Sample | FIG. | area (mm²) | (mm) | (mm) | Shape Clarity** | |
| Image 1 | FIG. 12 | 144 | 0.8 | 0.8 | 93 | A |
| Image 2 | FIG. 13 | 100 | 1.0 | 2.1 | 82 | A B C |
| Image 3 | FIG. 14 | 100 | 1.0 | 2.5 | 70 | C |
| Image 4 | FIG. 15 | 100 | 1.0 | 2.3 | 78 | B C |
| Image 5 | FIG. 16 | 100 | 1.0 | 3.6 | 45 | D |
| Image 6 | FIG. 17 | 100 | 1.0 | 5.1 | 28 | E |
| Image 7 | FIG. 18 | 100 | 1.0 | 4.0 | 46 | D |

*Distance: Distance between two adjacent apertures
**Levels not connected by same letter are significantly different.

Images having a distance between two adjacent apertures no longer than 3 mm were evaluated to provide better shape clarity in comparison with images having a distance between two adjacent apertures longer than 3 mm.

### Example 2. Virtual Diaper Image Preparation

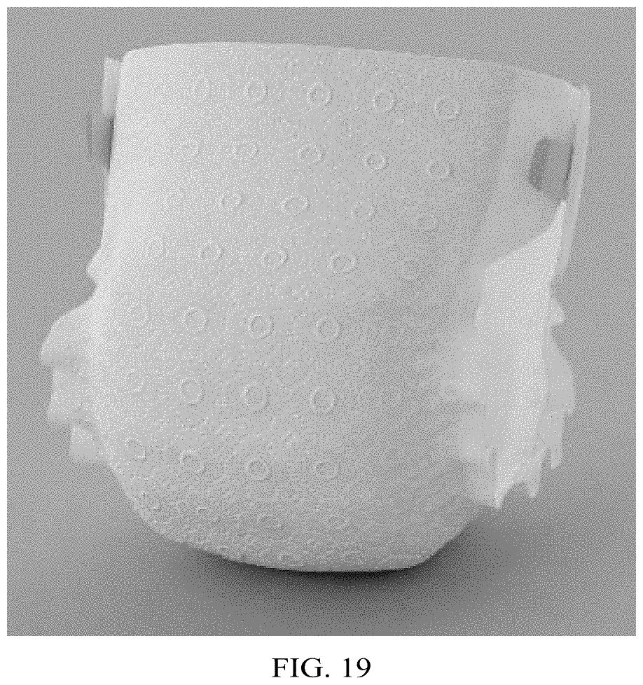

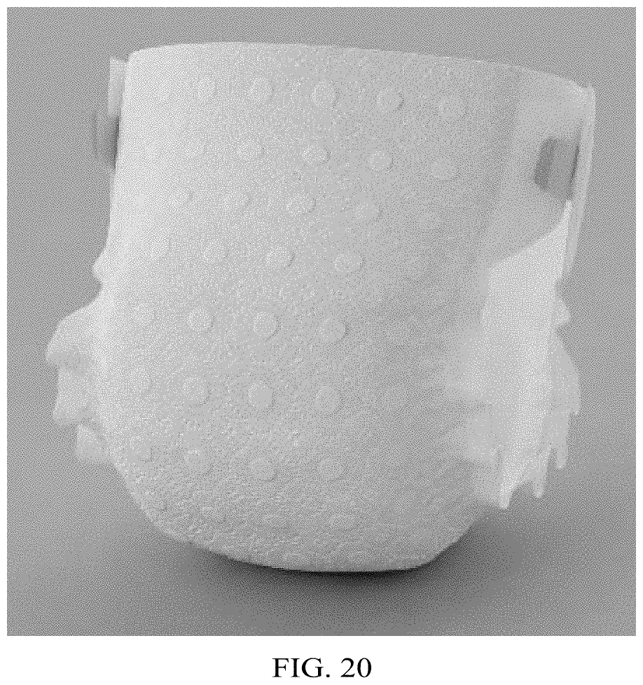

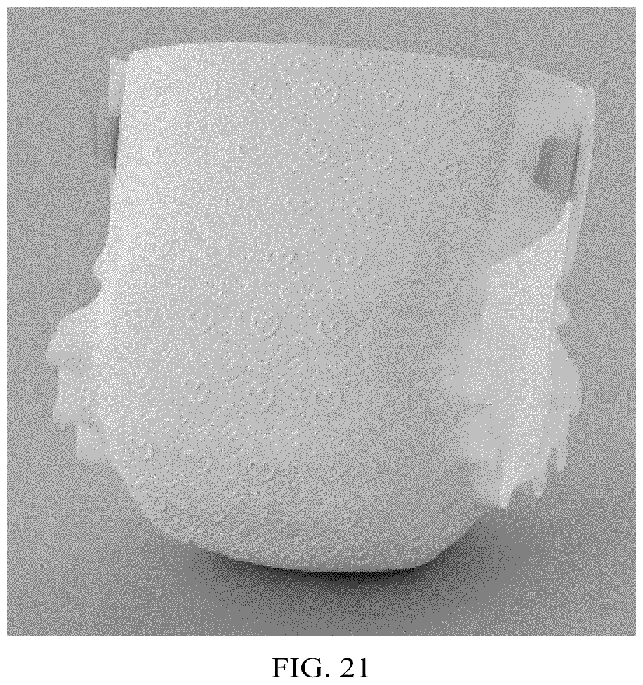

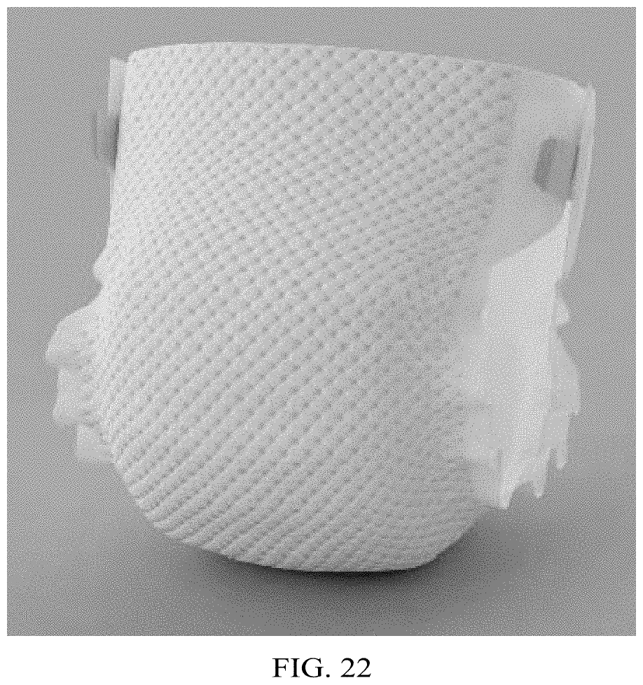

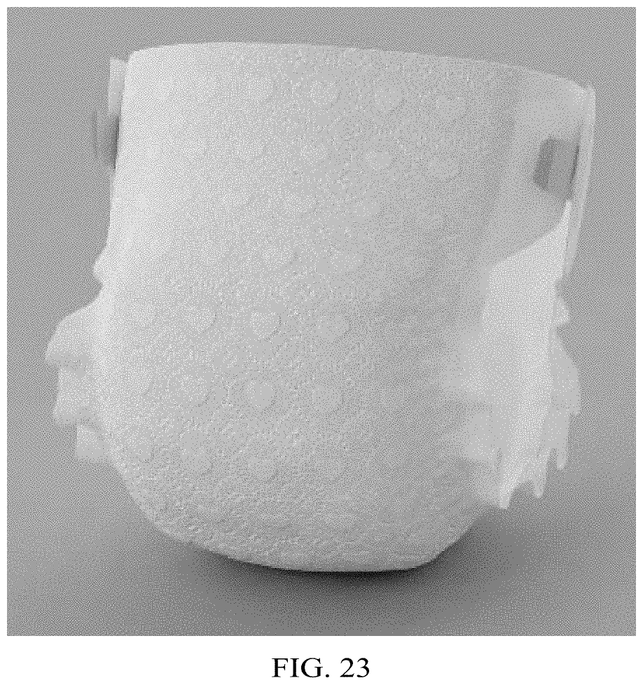

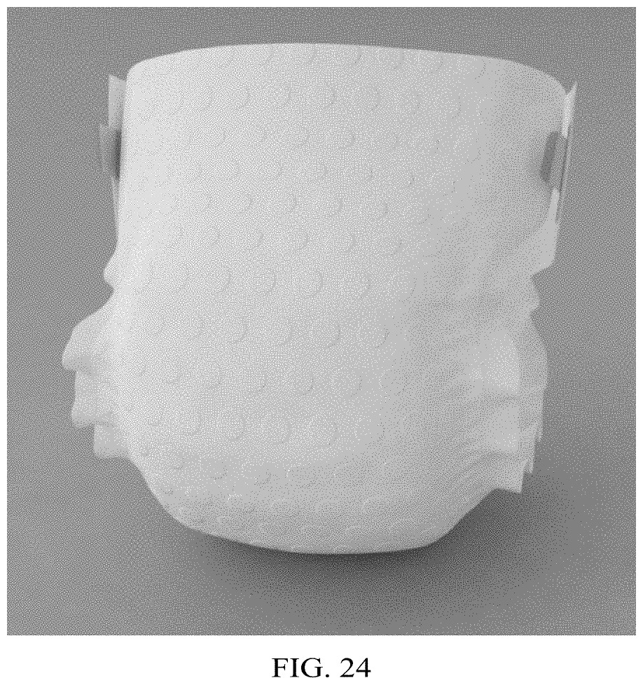

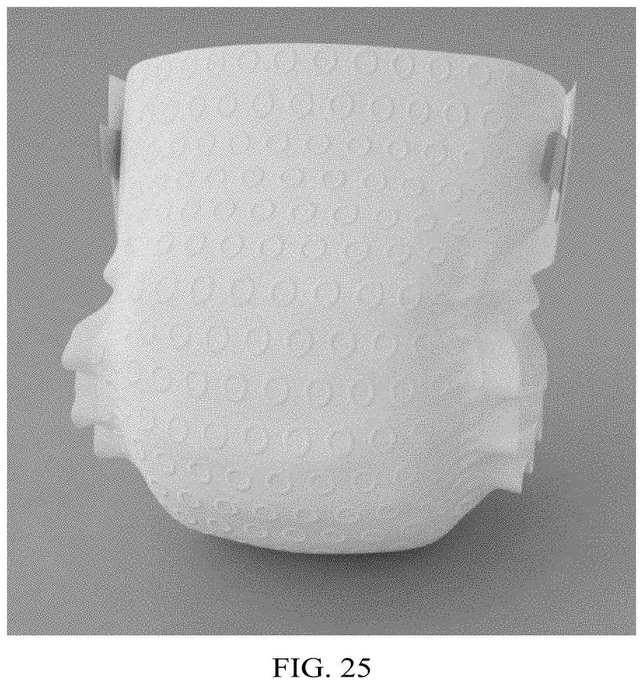

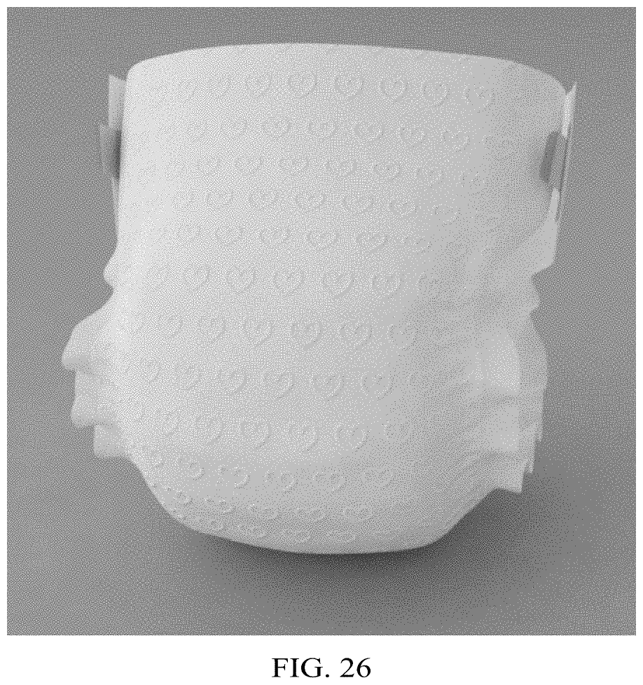

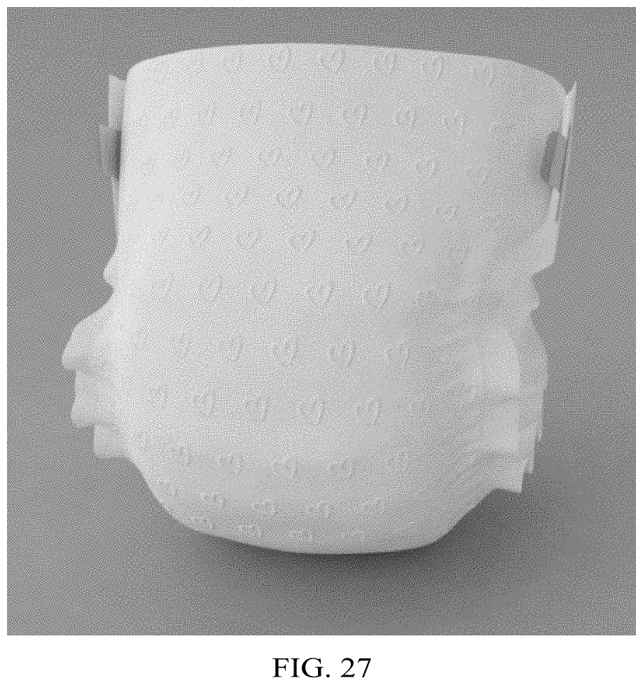

14 virtual diaper images having appearances of FIGS. 19-32 respectively, were prepared according to Diaper Photorealistic Image Generation under MEASUREMENT above.

### Example 3. Premium Impression Evaluation I

37 of premium diaper brand users with babies aged 0 to 36 months were recruited as panels for a virtual diaper appearance study. Computer-rendered images of diaper generated in Example 1 were tested. The panels were exposed to a group of 5 diaper images, Diaper 1-Diaper 5 having repeat patterns comprising protrusions in FIGS. 19-23 respectively, displayed one by one on a monitor at a distance of arm's length and requested to select 1) the most premium looking diaper and 2) most visibly patterned diaper. Numbers of selection of each diaper per each question were recorded as a score for the diaper. Results are indicated in Table 2 below.

TABLE 2

| | Dia- | Dia- | Dia- | Dia- | Dia- |
|---|---|---|---|---|---|
| | per 1 | per 2 | per 3 | per 4 | per 5 |
| Pattern | FIG. 19 | FIG. 20 | FIG. 21 | FIG. 22 | FIG. 23 |
| Max number of | 4 | 2 | 6 | 2 | 4 |
| transitions | | | | | |
| Number of | 0 | 0 | 1 | 0 | 1 |
| concave portions | | | | | |
| Premiumness | 2 | 1 | 27 | 2 | 5 |
| perception score* | | | | | |
| Pattern visibility | 4 | 12 | 11 | 6 | 4 |
| score* | | | | | |

Score*: number of selection

Diaper 4 having a repeat unit having a maximum number of transitions of 6 and a protrusion having one concave portion got a superior score on premiumness perception to other diapers tests. Interestingly, Diaper 2 having a repeat unit having a maximum number of transitions of 2 and a protrusion having no concave portion got the highest score with respect to obvious pattern visibility, however got the lowest score in respect with premium perception.

### Example 4. Premium Impression Evaluation II

18 of premium diaper brand users with babies aged 0 to 36 months were recruited as panels for a virtual diaper appearance study. Computer-rendered images of diaper generated in Example 1 were tested. The panels were exposed to a group of 7 diaper images, Diaper 6-Diaper 12 having repeat patterns comprising protrusions shown in FIGS. 24-30 respectively displayed all together on a high-definition super large screen, at a distance of about 1-2 meters, and requested to score premiumness of each diaper at 0-10 scales. Results are indicated in Table 3 below where the average score of 18 users is given for each diaper.

TABLE 3

| | Diaper 6 | Diaper 7 | Diaper 8 | Diaper 9 | Diaper 10 | Diaper 11 | Diaper 12 |
|---|---|---|---|---|---|---|---|
| Pattern | FIG. 24 | FIG. 25 | FIG. 26 | FIG. 27 | FIG. 28 | FIG. 29 | FIG. 30 |
| Max number | 2 | 4 | 6 | 6 | 6 | 4 | 4 |
| of transitions | | | | | | | |
| Number of | 0 | 0 | 1 | 1 | 1 | 1 | 4 |
| concave | | | | | | | |
| Average score | 5.28 | 5.39 | 7.39 | 8.06 | 8.33 | 7.94 | 8.00 |
| | | | Diapers | Diapers | Diapers 6, | Diapers | Diapers 6 |
| | | | 6 and 7* | 6 and 7* | 7 and 8* | 6 and 7* | and 7* |

*statistically difference at 95% confidence level compared letter.

Diapers 8-12 having repeat units comprising a maximum number of transitions of 4 or 6, and protrusions having at least one concave portion got a superior score on premiumness perception to diapers 6 and 7 which have repeat units having a maximum number of transitions less than 4 and/or protrusions having no concave portion.

Diapers 9 and 10 having protrusions in an asymmetric shape got higher scores than other diapers having symmetric protrusion. Diaper 10 having protrusions arranged in different orientations got a higher score than diaper 9 having the protrusion in the same shape as diaper 9, and the protrusions are arranged in the same orientation.

### Example 6. Premium Impression Evaluation III

18 of premium diaper brand users with babies aged 0 to 36 months were recruited as panels for a virtual diaper appearance study. Computer-rendered images of diaper generated in Example 1 were tested. The panels were exposed to a group of 3 diaper images, Diapers 8, 13 and 14 having repeat patterns comprising protrusions shown in FIGS. 26, 31 and 32 respectively, displayed on all together on a high-definition super large screen, at a distance of about 1-2 meters, and requested to evaluate premiumness of each diaper at 0-10 scales. Results are indicated in Table 4 below.

TABLE 4

| | Diaper 8 | Diaper 13 | Diaper 14 |
|---|---|---|---|
| Pattern | FIG. 26 | FIG. 31 | FIG. 32 |
| Max number of transitions | 6 | 6 | 6 |
| Number of concave | 1 | 1 | 1 |
| Space between 3D elements *: | 0.5 | 0 | 7.3 |
| (times length of a 3D element) | | | |
| Average score | 7.39 | 5.06 | 6.22 |

*: Space between two adjacent 3D elements, one in one repeat unit and the other in an adjacent repeat unit.

Diaper 8 having two adjacent protrusions spaced apart to each other about 0.5 time longer than a length of the protrusion got a higher score on premiumness perception than diaper 13 having two adjacent protrusions substantially contact to each other and diaper 14 having two adjacent protrusions spaced apart to each other about 7.3 times longer than a length of the protrusion. Under 95% confidence level, Diaper 8 was rated higher than diaper 13 with statistical significance.

### Example 7. Softness Hand Feel Evaluation

Three nonwovens, Nonwovens 1, 2 and 3 according to Table 5 were prepared. Eight females of age 20 to 40 were recruited as panels. Each sample of Nonwovens 1-3 in the size of 200 mm long by 150 mm wide was placed and taped onto a garment-facing surface of Size 4 (L-size) diapers sold in the tradename of “Pampers Hadaeno Ichiban Pants” (P&G) marketed in China, and blindly exposed to panels for touch feel evaluation. The panels ranked the diapers in view of softness feel. Average ranking results and non-aperture area are indicated in Table 5 below.

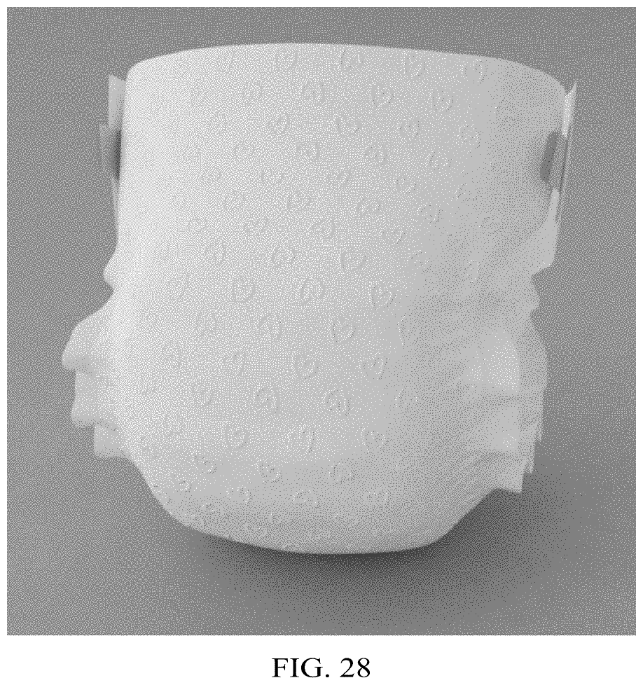

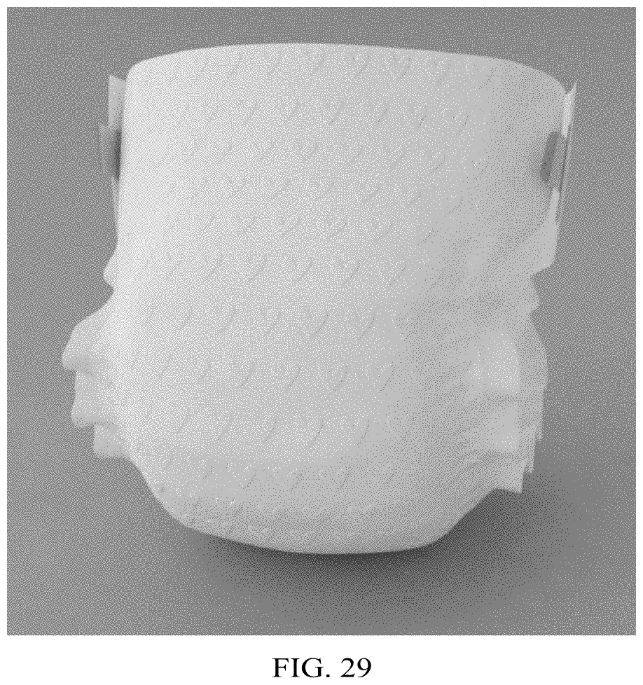

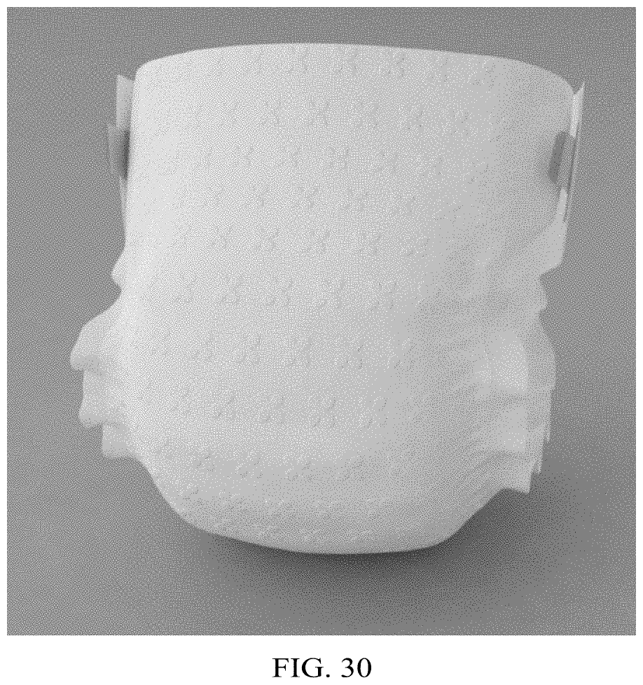

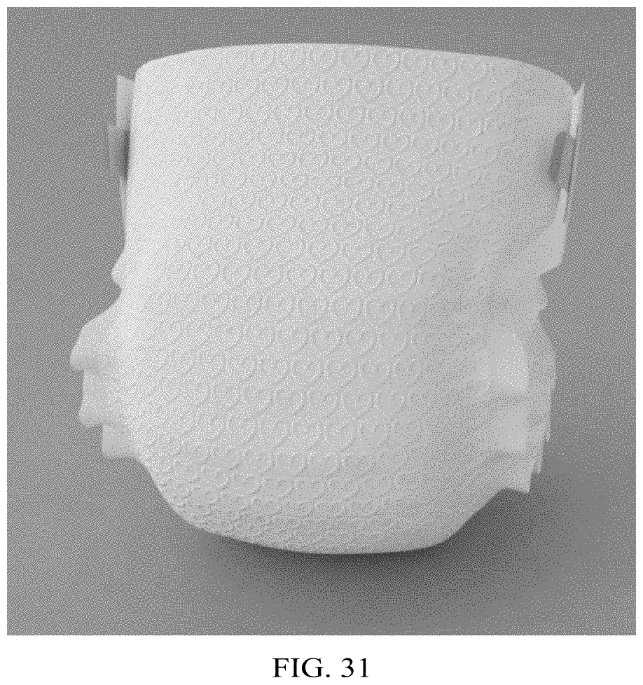

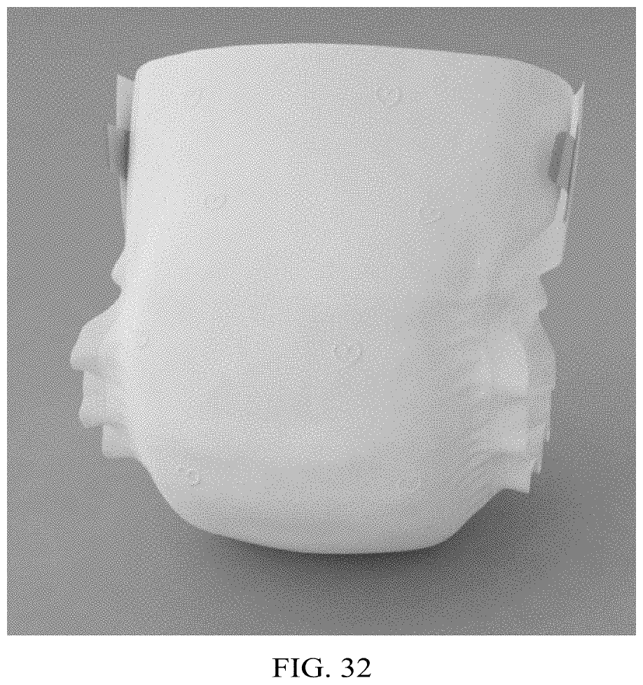

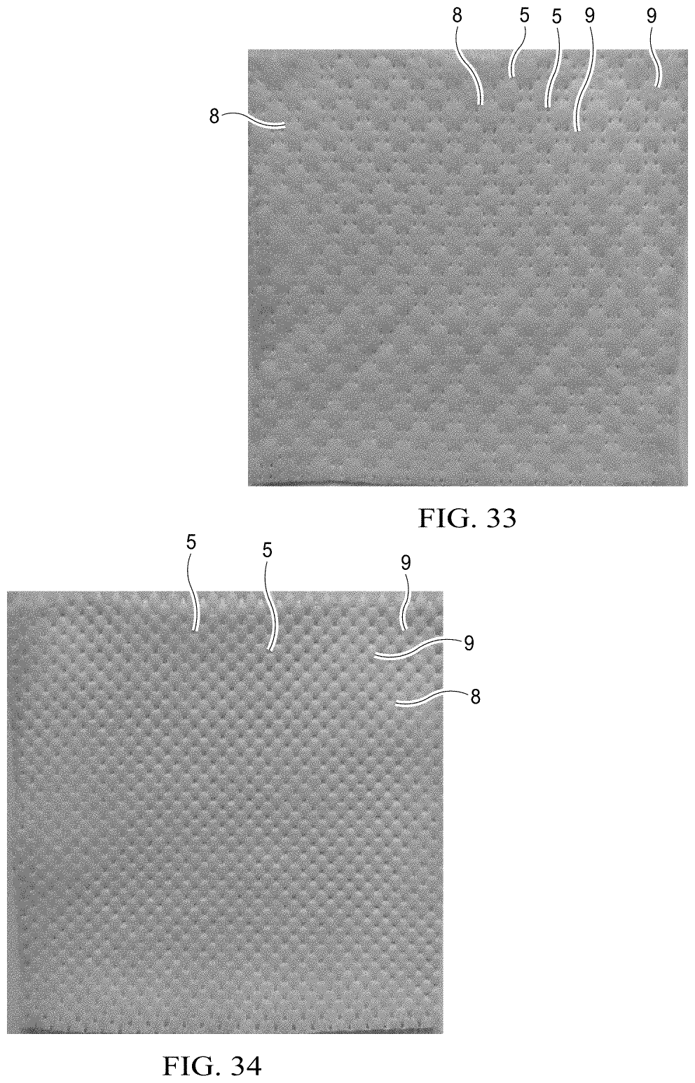

TABLE 5

| | Nonwoven 1 | Nonwoven 2** | Nonwoven 3*** |
|---|---|---|---|
| Nonwoven | 1^{st} layer: 50% 1.2 dpf | 1^{st} layer: 2 dpf | 1^{st} layer: 4 dpf |
| composition | PE/PET and 50% 2 dpf | PE/PET, 22 gsm | PE/PET, 15 gsm |
| | PE/PET, 21 gsm, | 2^{nd} layer: 2 dpf | 2^{nd} layer: 4 dpf |
| | 2^{nd} layer: 2 dpf | PE/PET, 55 gsm | PE/PET, 20 gsm |
| | PE/PE, 21 gsm | Adhesive: 3 gsm | 3^{rd} layer*: 2 dpf |
| | Adhesive: 3 gsm | | PE/PE, 21 gsm |
| Basis weight (g/m²) | 45 | 80 | 56 |
| Pattern | FIG. 1 | FIG. 33 | FIG. 34 |
| Area of non-aperture | 120 | 30 | 13 |
| area (mm²) | | | |
| No. of apertures in a | 40 | 8 | 4 |
| repeat unit | | | |
| % selected rank 1 | 50.0 | 25.0 | 25.0 |
| % selected rank 2 | 37.5 | 62.5 Nonwoven 3* | 0.0 |
| % selected rank 3 | 12.5 | 12.5 | 75.0 Nonwoven 1 |
| | | | and Nonwoven 2* |

*statistically difference at 95% confidence level compared letter.
Nonwoven 2**: Laminate nonwoven having aperture 5, land 8 and protrusion 9. The first layer is not bonded to the second layer in the protrusion.
Nonwoven 3***: Laminate nonwoven having aperture 5, land 8 and protrusion 9. A precursor nonwoven laminate of the first layer and the second layer having a pattern of FIG. 34 where the first layer and the second layer were bonded with each other in the entire precursor nonwoven laminate was prepared, and the third layer was added below the second layer of the precursor nonwoven laminate.

Nonwoven 1 having more than 100 mm² of an area of non-apertured area was selected as a top softness feel ranking among 3 tested nonwovens and significantly less selected as a bottom ranking compared with nonwoven 3 which has least non-apertured area.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A three-dimensional substrate comprising a pattern comprising a repeat unit,
wherein the repeat unit comprises a non-apertured area and a plurality of apertures defining the non-apertured area,
wherein the non-apertured area comprises a protrusion and a land area defining the protrusion, and
wherein the non-apertured has an area no less than 100 mm², as measured according to Area of Non-apertured Area under Measurement.

2. The three-dimensional substrate of claim 1, wherein the protrusion comprises at least one concave portion.

3. The three-dimensional substrate of claim 1, wherein the repeat unit has at least 6 of a maximum transition numbers, as measured according to Protrusion—Land Transition.

4. The three-dimensional substrate of claim 1, wherein the land area is substantially flat.

5. The three-dimensional substrate of claim 1, wherein the three-dimensional substrate comprises a nonwoven layer.

6. The three-dimensional substrate of claim 1, wherein the plurality of apertures comprises at least 20 apertures.

7. The three-dimensional substrate of claim 1, wherein a distance between two adjacent apertures is no more than about 3 mm.

8. The three-dimensional substrate of claim 1, wherein the land area is substantially surrounded by the apertures.

9. The three-dimensional substrate of claim 1, wherein the three-dimensional substrate comprises a first layer and a second layer.

10. The three-dimensional substrate of claim 9, wherein the first layer comprises the protrusion where the first layer does not bond to the second layer.

11. The three-dimensional substrate of claim 1, wherein the repeat unit repeats in a first direction.

12. The three-dimensional substrate of claim 11, wherein the repeat unit repeats in a second direction, the second direction being different from the first direction.

13. The three-dimensional substrate of claim 1, wherein two adjacent protrusions in the substrate are spaced apart from each other at least 0.5 times a length of one of the two protrusions.

14. The three-dimensional substrate of claim 5, wherein the three-dimensional substrate comprises natural fibers.

15. The three-dimensional substrate of claim 1, wherein the three-dimensional substrate comprises a colored region.

16. An absorbent article comprising:
a liquid pervious topsheet,
a liquid impervious backsheet,
an absorbent core disposed between the topsheet and the backsheet, and
a three-dimensional substrate according to claim 1.

17. The absorbent article of claim 16, wherein the three-dimensional substrate is joined to the liquid impervious backsheet such that the three-dimensional substrate forms a garment facing surface of the article.

18. The absorbent article of claim 16, wherein the liquid pervious topsheet comprises the three-dimensional substrate such that the three-dimensional substrate is disposed at a wearing facing surface of the article.
